(19) United States
(12) Patent Application Publication
(10) Pub. No.: US 2004/0241867 A1
(43) Pub. Date: Dec. 2, 2004

(54) Method of analyzing a wafer for metal impurities

(76) Inventors: Mark L. Jones, Portland, OR (US); Ansgar Koch, Bavaria (DE); Fumitoshi Ito, Lake Oswego, OR (US); James J. Shen, Portland, OR (US)

Correspondence Address: KOLISCH HARTWELL, P.C., 520 S.W. YAMHILL STREET, SUITE 200, PORTLAND, OR, 97204, US

(21) Appl. No.: 10/760,661

(22) Filed: Jan. 15, 2004

Related U.S. Application Data
(60) Provisional application No. 60/440,991, filed on Jan. 17, 2003.

(51) Int. Cl. G01N 33/20

(52) U.S. Cl. 436/73

(57) ABSTRACT

A method for quantitatively analyzing a wafer for metal impurities is disclosed, wherein the wafer has first and second surfaces. The method includes heating the first surface of the wafer to diffuse the metal impurities to the second surface in an environment at least substantially free of contamination, cooling the first surface of the wafer, and quantitatively analyzing the second surface of the wafer for the metal impurities.

### CROSS-REFERENCE TO RELATED APPLICATIONS

This application claims priority under 35 U.S.C. 119(e) to U.S. Provisional Patent Application Ser. No. 60/440,991 entitled “Method of Analyzing a Wafer for Metal Impurities,” filed Jan. 17, 2003, the disclosure of which is hereby incorporated by reference.

### TECHNICAL FIELD

The present invention relates to a method of analyzing a semiconductor wafer for the presence of bulk metal impurities via surface techniques.

### BACKGROUND OF THE INVENTION

The presence of even very low levels of impurities, such as nickel and other metals, in a silicon wafer can degrade the electrical characteristics of the wafer. Thus, it is important to be able to determine the presence and concentrations of these impurities in the bulk regions of wafers before the wafers are used to make devices to ensure the electrical characteristics of the wafer match those required by the device specifications.

Almost as important as determining the presence and concentrations of these metals is the ability to determine their presence and concentrations in a quick, efficient and reliable manner. The longer a sample analysis takes, the longer a shipment of a batch of wafers to a customer may be held up. Furthermore, slower analytical techniques may be unsuitable for use in monitoring a wafer manufacturing process to identify potential sources of metal contamination.

Impurity levels in wafers are typically determined via several different techniques. For example, dry techniques, such as Dynamic Secondary Ion Mass Spectrometry (Dynamic SIMS), may be used for some types of analyses. However, the detection limits of these methods, typically on the order of 10¹⁴-10¹⁵ atoms/cm³ for copper and nickel, may not be low enough for accurate process monitoring and quality control for high-purity wafers.

Wet chemical techniques may be used for some analyses as well. For example, some wet chemical methods involve digesting a small sample of silicon in a concentrated nitric acid and hydrofluoric acid solution, and then evaporating the solution to recover impurities for analysis. Several different methods may be used for digesting the sample, including hotplate methods and microwave methods. These methods may provide low detection levels, but also may be relatively slow.

Another wet chemical technique involves allowing a wafer to sit for a time period of approximately 30-60 days after fabrication to allow nickel and other contaminants to diffuse to the wafer surfaces. The surfaces are then decomposed with HF, and metals on the surface are extracted for analysis. As with the digestion methods, the so-called “long-term-storage” method may provide low detection limits, but may also be slow, taking as long as 30-60 days to prepare a wafer for analysis. Once the analysis has been performed, the batch of wafers from which the test wafer was taken may have already been shipped to a customer, making this method ineffective as a quality control measure.

Additionally, wafers have been analyzed for copper using a technique in which the wafer is heated to cause copper to diffuse to the wafer surface for analysis. Examples of these methods are described in M. B. Shabani, Kinetic of Low-Temperature Out-Diffusion of Cu from Silicon Wafers, Abstract No. 1389, submitted to ECS in October 1999, and Shabani, M., and Okuuchi, S., Effect of Dopants and Oxygen Precipitation on Low-Temperature Out-Diffusion and Gettering of Cu in Silicon Wafer, Proceedings of Seventh International Symposium on Silicon-on-Insulator Technology and Devices, published by The Electrochemical Society, 96-3, 10 (1997). Other relevant studies are described in C. M. McCarthy et al., Room Temperature Copper Out-Diffusion from the Bulk of Silicon Wafers and its Behavior Following Various Postanneal Treatments, ECS Proc. 98-1, 629-640 (1998). However, these references only describe the analysis of wafers for copper, and not for other metals. This may be due to the fact that other metals have different diffusion characteristics in silicon than copper, and thus may be affected in different ways by the presence of impurities in the silicon than copper.

### SUMMARY

One embodiment provides a method for quantitatively analyzing a wafer for metal impurities, wherein the wafer has first and second surfaces. The method includes heating the first surface of the wafer to diffuse the metal impurities to the second surface in an environment at least substantially free of contamination, cooling the first surface of the wafer, and quantitatively analyzing the second surface of the wafer for the metal impurities.

### BRIEF DESCRIPTION OF THE DRAWINGS

FIG. 1 is a flow diagram of a method of analyzing a wafer for metal impurities according to one embodiment of the present invention.

FIG. 2 is a plot comparing signal-to-noise ratios of nickel determinations from a plurality of samples prepared by the method of FIG. 1 using different experimental conditions.

FIG. 3 is a plot showing the results of nickel determinations performed on a plurality of wafers before and after preparing the wafers for analysis via optimum experimental conditions determined from the plot of FIG. 2.

FIG. 4 is a plot showing the results of nickel determinations performed on a series of wafers taken from a single manufacturing batch and heated to different temperatures.

FIG. 5 is a plot showing the results of baseline nickel determinations performed on a single production wafer over a series of days.

### DETAILED DESCRIPTION OF THE DEPICTED EMBODIMENTS

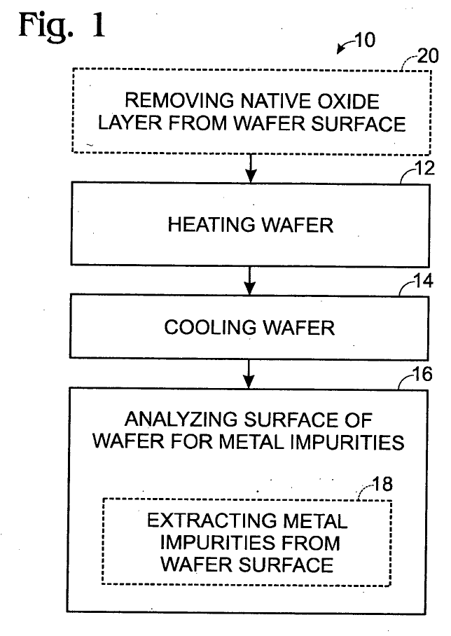

FIG. 1 shows, generally at 10, a method of analyzing a wafer for metal impurities according to one embodiment of the present invention. Method 10 is typically performed on one or more test wafers from a batch of wafers after wafer processing has been substantially completed, but may also be performed at any other desired stage in a wafer manufacturing process. Method 10 has been found to be particularly applicable to the analysis of wafers for the presence and concentrations of bulk and surface nickel, but may be used in the analysis of other impurities as well. Moreover, method 10 may be used to remove nickel contamination from wafers before shipping the wafers to customers, as described in more detail below.

Method 10 includes first heating the wafer via a heat source, such as a hot plate or similar device at 12, and then cooling the wafer at 14. After cooling the wafer, the surface is analyzed for nickel content at 16. The step of analyzing the wafer surface for impurities may include examining the wafer surface directly for impurities, for example by total x-ray fluorescence (TXRF). Alternatively, the step of analyzing the wafer surface for impurities may include extracting the impurities from the wafer surface via wet chemical techniques, as indicated in dashed lines at 18, and then analyzing the extracted sample for impurities, for example, by inductively coupled plasma-mass spectrometry (ICP-MS) or graphite furnace atomic absorption spectroscopy (GFAAS). Method 10 also may include removing a native oxide layer from the wafer surface before heating the wafer, as indicated at 20. Each of these steps is described in more detail below.

Heating the wafer via the hot plate causes nickel in bulk regions of the wafer to diffuse to the surface of the wafer opposite the hot plate. This is in contrast to commonly used nickel analytical methods known as “long-term-storage” methods, in which a wafer is allowed to sit in storage for a sufficient period of time for nickel contaminants to diffuse from the wafer bulk to the surface. The above-referenced papers describe a heating method for the determination of copper. However, such an effect has not been described for nickel. As discussed above, this is at least partially due to the fact that nickel has different diffusion characteristics in silicon than copper, and is affected in different ways by the presence of impurities in the silicon than copper. Furthermore, the use of the hotplate to diffuse copper to the surface of a wafer is described in some of these references as difficult to use in a quantitative manner, which again may be due to the different diffusivity characteristics of copper in the presence of different wafer impurities. However, the techniques disclosed herein may allow bulk nickel to be reproducibly quantified, as described in more detail below.

As mentioned above, the native oxide layer on the wafer surface either may or may not be removed before the wafer is heated. However, removal of the native oxide from the wafer has been found to improve the sensitivity of the nickel analysis in some situations. The native oxide layer may be removed from the wafer surface in any suitable manner, for example, by vapor phase decomposition (VPD). This technique involves placing the wafer in an enclosed space, and exposing the wafer to HF vapor for a suitable duration of time, typically 8-10 minutes. The native oxide is removed as SiF₄ vapor and H₂O. Alternatively, the native oxide may be removed by physical methods.

The native oxide layer may be removed from both faces of the wafer, or only one face of the wafer. Where a hotplate is used to heat the wafer, the hotplate may cause nickel to diffuse to the opposite surface of the wafer as the hotplate. Thus, in this case, it may be desirable to remove the native oxide layer only from the surface where nickel analysis or extraction will occur if desired. While removal of the native oxide layer is shown as preceding the heating of the wafer in FIG. 1, it will be appreciated that the native oxide removal also may occur during the heating of the wafer if desired. In this case, the native oxide removal may be performed while the wafer is heating, or even once the wafer has reached its final temperature for step 12 of method 10.

Any suitable heating device may be used to heat the wafer at 12 to cause diffusion of nickel to the wafer surface. Examples include, but are not limited to, ovens, furnaces and tube furnaces. Where a hotplate is used to heat the wafer, any suitable type of hotplate may be used. Examples include, but are not limited to, those with a ceramic heating surface.

The wafer may be placed directly on top of the hotplate, or the hotplate may be provided with a susceptor to hold the wafer. The use of a susceptor may offer some advantages over placing the wafer directly on the hotplate. For example, the susceptor may protect the hotplate surface from HF condensation from the wafers. Also, the susceptor may help prevent the wafers from moving during heating, may help to improve temperature uniformity across the wafer, and may provide a cleaner metal-free surface than the hotplate surface.

The wafers may be heated to any desired temperature. Typically, the wafer is heated to a temperature between 200 and 500 degrees Celsius, although temperatures outside this range may be used as well. It has been found that the use of lower temperatures may help to increase the rate of nickel diffusion. Likewise, the wafer may be heated for any desired time duration. For example, it may be desirable to heat the wafer for a relatively short period of time so that the nickel analysis may be completed more quickly and efficiently. Longer heating times may increase the amount of nickel that diffuses to the wafer surface, but also may lower throughput. Examples of suitable heating times include, but are not limited to, times of 0.5-4 hours, and more typically times of 0.5-3 hours.

Heating typically is performed in an environment at least substantially free of contamination. For example, the wafer may be heated in a suitable cleanroom environment, such as under a Class 10 laminar flow hood. Alternatively, the wafer may be heated in a dry or non-oxygen-containing environment, such as a nitrogen gas environment, to avoid re-oxidizing the wafer surface. Additionally, the use of a susceptor, as described above, may contribute in achieving a suitable heating environment by preventing HF condensation and providing a cleaner metal-free surface than the hotplate surface.

After heating the wafer for a desired time period, the wafer is cooled. The wafer may be cooled in any desired manner, and at any desired rate. Where fast cooling is desired, the wafer may be cooled by transferring the wafer from the hotplate (or susceptor or other wafer holder) to a suitable cooling plate (or other surface) with a lower temperature than the heated wafer. Alternatively, the wafer may be transferred to a cool or cold solution of a suitable fluid, such as ethylene glycol followed by a bath in a solvent such as isopropyl alcohol or the like. Where slow cooling is desired, the wafer may be left with the hotplate or susceptor, and allowed to return gradually to room temperature.

Once the wafer is cooled, the wafer surface is analyzed to determine the surface concentration of nickel. Any suitable method of analysis may be used. For example, TXRF may be used to measure the nickel on the surface directly, without any wet chemical extraction steps. However, there is a possibility that nickel may not be distributed evenly across the surface, and thus that this measurement may be inaccurate.

Alternatively, as indicated at 18 in FIG. 1, the nickel may first be extracted from the surface of the wafer before being analyzed. This extraction may be performed by any suitable method. One example of a suitable extraction method is first decomposing the surface of the wafer using VPD as described above, and then scanning the surface of the wafer with a droplet of deionized water containing dilute concentrations of HF and H₂O₂ to extract surface nickel from the exposed silicon surface layer. The surface of the wafer after VPD is hydrophobic, so the surface tension of the fluid droplet holds the droplet together. Next, a hollow stylus is inserted into the droplet to hold the droplet via capillary action, and the stylus is moved to scan the droplet across the wafer surface. Exposed nickel is oxidized by the fluid and dissolves into the fluid. The droplet may then be collected for analysis by such methods as ICP-MS, GFAAS, VPD-ICP-MS, or VPD-TXRF. Alternatively, a plate method of extraction may be used wherein a droplet of a similar extraction solution is added to the wafer, and then spread across the top of the wafer in a thin layer by the application of pressure with a smooth, hydrophobic plate.

Before performing quantitative analysis, it may be desirable to optimize the sample preparation and extraction method. The method may be optimized in any suitable manner, including, but not limited to, statistical methods such as the Taguchi method. An exemplary Taguchi optimization method is as follows. First, preparation variables to be optimized are selected. Some exemplary variables that may be optimized include heating time, heating temperature, cooling conditions, and whether the wafer surface is subjected to VPD before heating.

Next, for each variable to be optimized, test values are selected. Table 1 lists exemplary test values to be evaluated for each preparation variable.

TABLE 1

| Factor | Test Value 1 | Test Value 2 | Test Value 3 |
|---|---|---|---|
| Temperature (° C.) | 300 | 400 | 500 |
| VPD before | Yes | No | Yes |
| heating? | | | |
| Heating time (hr) | 0.5 | 2 | 3 |
| Cooling rate | Fast | Slow | Slow |

A “fast” cooling rate signifies the transfer of the wafer from the hotplate to a cooling plate, while a “slow” cooling rate signifies turning the hotplate off and allowing the wafer to cool gradually to room temperature.

The optimal value for each test variable may be determined by preparing a series of test wafers with a similar nickel contamination level via the preparation methods listed in Table 2, and then analyzing each test wafer for nickel to determine a signal-to-noise ratio for each analysis.

TABLE 2

| Experiment | Temperature | VPD before | | |
|---|---|---|---|---|
| No. | (° C.) | heating? | Time (hr) | Cooling rate |
| 1 | 300 | Yes | 0.5 | Fast |
| 2 | 300 | No | 2 | Slow |
| 3 | 300 | Yes | 3 | Slow |
| 4 | 400 | Yes | 2 | Slow |
| 5 | 400 | No | 3 | Fast |
| 6 | 400 | Yes | 0.5 | Slow |
| 7 | 500 | Yes | 3 | Slow |
| 8 | 500 | No | 0.5 | Slow |
| 9 | 500 | Yes | 2 | Fast |

Each preparation may be repeated for more than one wafer. After completing the preparations, the wafers are analyzed as described above for nickel. The signal-to-noise ratio is determined for each analysis, and the signal-to-noise ratios of analyses of multiple wafers that were prepared via the same preparation method are averaged. Finally, the signal-to-noise ratios are plotted to determine which variables produce the highest signal-to-noise ratio using known methods of statistical analysis to determine the optimum tested values of the selected sample preparation variables. Preferably, analysis takes place no longer than approximately 24 hours after hotplate treatment to avoid nickel diffusing back into the silicon.

The optimization routine was performed on a series of four wafers for each experiment, for a total of 36 wafers tested. The wafers were not intentionally contaminated with nickel, but were known to have relatively high concentrations of nickel. The signal-to-noise ratios of these experiments are listed in Table 3.

TABLE 3

| Experiment | | | | |
|---|---|---|---|---|
| No. | First wafer | Second wafer | Third wafer | Fourth wafer |
| 1 | 0.60 | 0.60 | 1.20 | 0.60 |
| 2 | 0.50 | 0.50 | 0.50 | 0.50 |
| 3 | 23.60 | 20.70 | 36.50 | 2.30 |
| 4 | 10.40 | 13.60 | 8.30 | 1.30 |
| 5 | 0.50 | 0.50 | 0.50 | 0.50 |
| 6 | 8.80 | 2.10 | 4.10 | 0.70 |
| 7 | 1.20 | 0.50 | 4.50 | 0.50 |
| 8 | 0.50 | 0.50 | 0.50 | 0.50 |
| 9 | 0.50 | 0.50 | 0.50 | 1.44 |

The optical conditions may be selected based upon those that produce the larger signal-to-noise ratios.

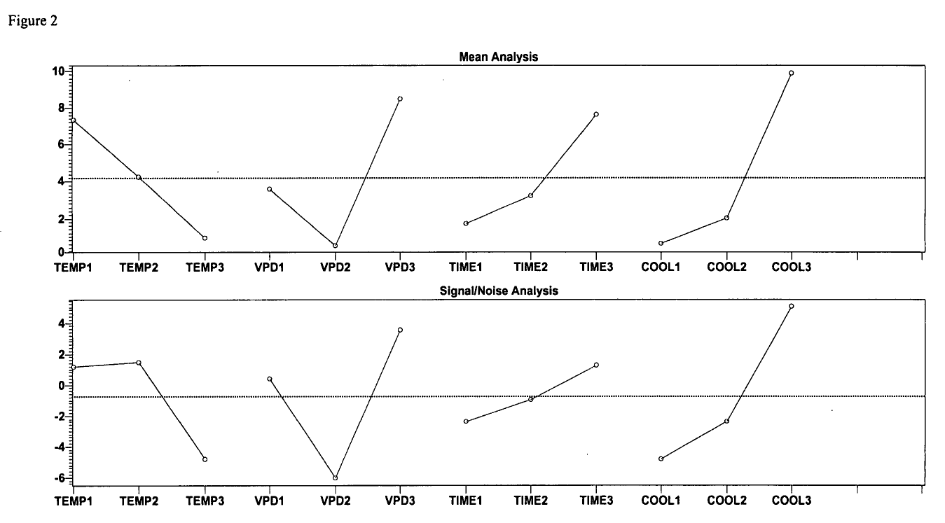

FIG. 2 shows plots of the signal-to-noise data for each test value of each selected preparation variable generated by analyzing the variation of the data from the above experimental data. As is evident in the graph, the best signal-to-noise ratios were found where the temperature was 300 degrees Celsius, the native oxide was removed via VPD before heating, the wafer was heated for 3 hours, and the wafer was cooled slowly. It will be appreciated that these optimum values may apply only to nickel determination in wafers with particular dopants, and that wafers with other dopants, or analyses for metals other than nickel or more than one metal, may have different optimum conditions that may require separate characterization.

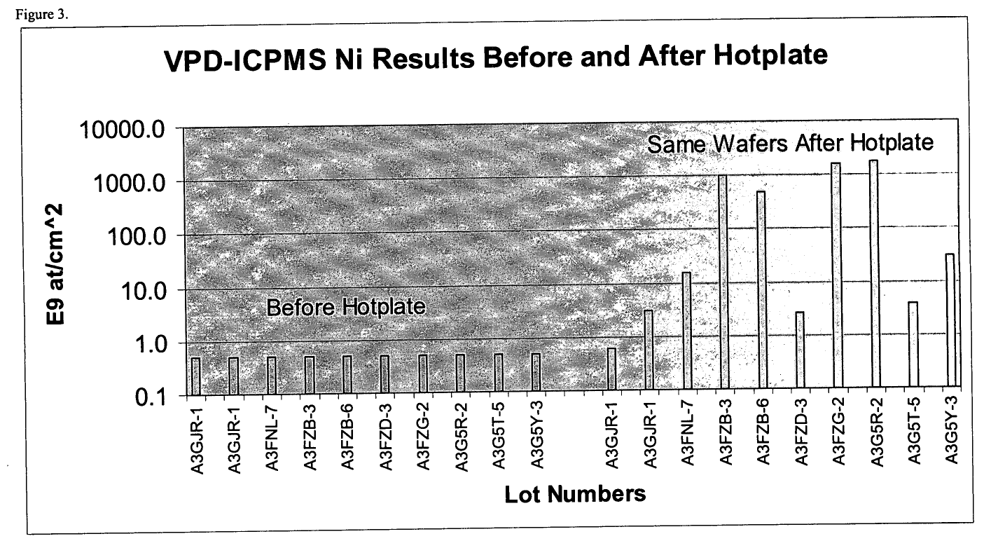

Next, a series of wafers known to have a problem with metals contamination were tested for nickel contamination. The wafers were single-side polished, 200 mm diameter, boron-doped, with a resistivity between 0.005-0.01 ohms-cm, not epi, with an acid-etched backside. The wafers were analyzed within a few days of production. FIG. 3 shows a plot of the results of these determinations. Each wafer was tested for nickel twice—once before heating the wafer according to method 10, and once after. The wafers were prepared using the optimum conditions determined from the plot of FIG. 2: the native oxide was removed from the wafers via VPD, the wafers were heated to 300 degrees C. for three hours, and then cooled slowly.

The results from the nickel determinations performed before native oxide removal, heating and cooling are shown on the left side of the graph of FIG. 3, and the results of the determinations performed after these steps are shown on the right side of the graph. The results from the pre-treated wafers seem to indicate that little or no nickel is present in the wafers. However, the results after treatment indicate that a fairly significant quantity of nickel, on the order of 10¹² atoms of Ni/cm², diffused to the surface of some of the wafers, and thus indicate that the bulk concentration of nickel may be significantly higher in these wafers than the pre heat-treatment determination results indicate. It will be appreciated that this figure may be converted into a bulk nickel measurement by multiplying the determined surface concentration of nickel by the wafer thickness.

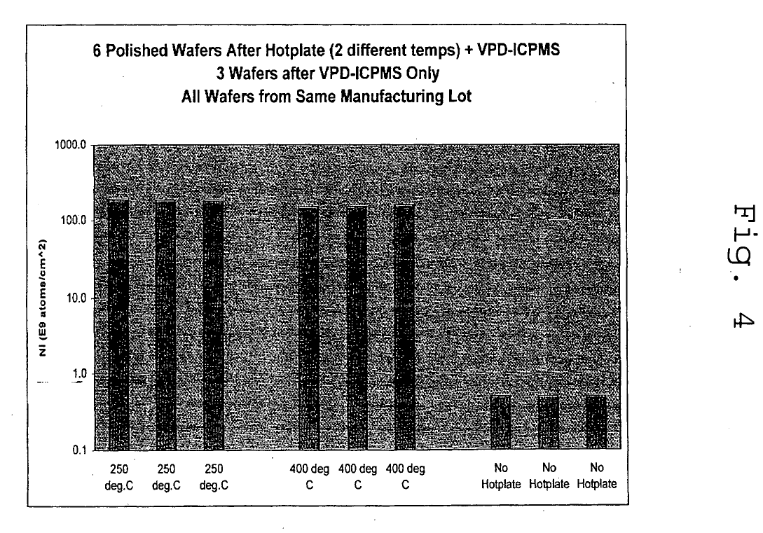

FIG. 4 shows the results of another nickel determination performed on a set of wafers taken from the same manufacturing batch. Some wafers were not heated, others were heated to 250 degrees Celsius, and others were heated to 400 degrees Celsius. As with the results shown in FIG. 3, FIG. 4 shows that heating the wafer helps to cause diffusion of nickel to the surface of the wafer, and thus improves the sensitivity of nickel determinations. Moreover, FIG. 4 shows that slightly more nickel diffused to the surface at 250 degrees Celsius than at 400 degrees Celsius, indicating that the use of a lower temperature heating cycle may increase the rate of nickel diffusion.

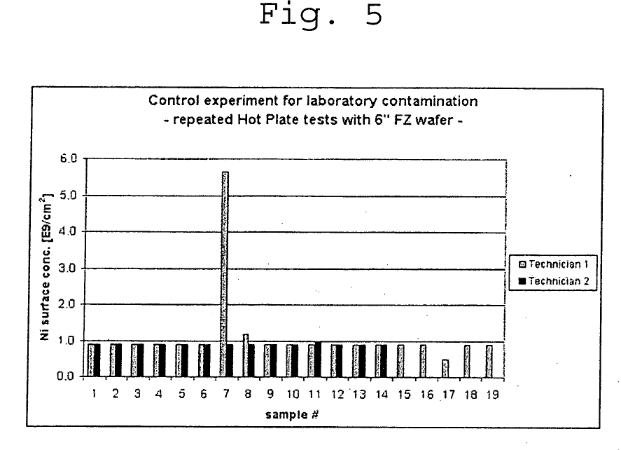

As mentioned above, method 10 has been found to allow nickel concentrations to be reproducibly determined. FIG. 5 shows the results of a series of measurements performed on a wafer taken from the production line at a rate of two measurements each day (one measurement each day by each of two technicians). As is evident in the graph, method 10 is shown to produce very consistent results, with only a single anomalous data point for the approximately 33 data points measured. This indicates that method 10 may be used to reproducibly quantify nickel concentrations in wafers over a period of time.

The determination of total nickel in a wafer may require calibration curves to be calculated for accurate quantitative analysis. A calibration curve may be calculated as follows. Calibration may require the use of a wafer with a well-known and controlled level of nickel contamination. Such a wafer may be fabricated by the spin-on of a standard solution of Ni, followed by a high-temperature anneal to drive the nickel into the wafer. Alternatively, the nickel may initially be added by backside ion implantation before annealing.

The amount of nickel that migrates to the wafer surface during heating may vary depending upon such factors as the resistivity of the wafer, the type and concentration of dopants in the wafer, the presence of an external gettering layer (such as a polysilicon backside layer), etc. For this reason, it may be desirable to determine a calibration curve for each type of wafer to be tested.

Method 10 offers significant advantages over other methods of nickel determination in wafers. For example, a complete analysis via method 10 may be performed so quickly that the method may be used to monitor nickel contamination during the manufacturing process, as well as to test wafer batches for contamination before shipping to a customer. In contrast, both digestion methods and long-term-storage methods take much longer to perform, and thus are not suitable for use in monitoring contamination during wafer manufacturing processes. Furthermore, as mentioned above, the method may also be integrated into a wafer production process to help reduce nickel contamination levels. This may be accomplished by simply adding a rinse step in place of the extraction step of method 10 to remove diffused nickel from the wafer surface before performing final steps of the production process.

While the embodiments described above involve varying the heating temperature, the heating time, pre-heating surface treatments, and cooling conditions of the wafers, it will be appreciated that other preparation variables, such as the composition of the ambient atmosphere during heating, the rate at which the temperature of the wafer is increased during heating, the chemical composition of the droplet used to extract the nickel from the surface of the wafer, etc. may be optimized as desired. Also, while the embodiments above are described in the context of determining the presence and concentration of nickel in wafers, it will be appreciated that the method may also be applied to other metals, such as copper and other transition metals.

Furthermore, although the present disclosure includes specific embodiments, specific embodiments are not to be considered in a limiting sense, because numerous variations are possible. The subject matter of the present disclosure includes all novel and nonobvious combinations and subcombinations of the various elements, features, functions, and/or properties disclosed herein. The following claims particularly point out certain combinations and subcombinations regarded as novel and nonobvious. These claims may refer to “an” element or “a first” element or the equivalent thereof. Such claims should be understood to include incorporation of one or more such elements, neither requiring nor excluding two or more such elements. Other combinations and subcombinations of features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

We claim:

1. A method for quantitatively analyzing a wafer for metal impurities, wherein the wafer has first and second surfaces, comprising:
heating the first surface of the wafer to diffuse the metal impurities to the second surface in an environment at least substantially free of contamination;
cooling the first surface of the wafer; and
quantitatively analyzing the second surface of the wafer for the metal impurities.

2. The method for quantitatively analyzing a wafer of claim 1, wherein the metal impurities include nickel.

3. The method for quantitatively analyzing a wafer of claim 1, wherein heating the first surface of the wafer includes heating the first surface of the wafer via a hotplate.

4. The method for quantitatively analyzing a wafer of claim 3, wherein heating the first surface of the wafer via a hotplate includes using a susceptor between the first surface of the wafer and the hotplate.

5. The method of quantitatively analyzing a wafer of claim 3, wherein cooling the first surface of the wafer includes switching off the hotplate and leaving the first surface of the wafer in thermal communication with the hotplate.

6. The method of quantitatively analyzing a wafer of claim 1, wherein heating the first surface of the wafer includes heating the wafer in a non-oxygen containing environment.

7. The method of quantitatively analyzing a wafer of claim 6, wherein heating the wafer in a non-oxygen containing environment includes heating the wafer in a nitrogen gas environment.

8. The method of quantitatively analyzing a wafer of claim 1, wherein cooling the first surface of the wafer includes cooling the wafer via a cooling plate.

9. The method of quantitatively analyzing a wafer of claim 1, wherein cooling the first surface of the wafer includes cooling the wafer via a fluid solution.

10. The method of quantitatively analyzing a wafer of claim 9, wherein the fluid solution includes ethylene glycol.

11. The method of quantitatively analyzing a wafer of claim 1, wherein quantitatively analyzing the second surface of the wafer includes measuring the metal impurities on the second surface.

12. The method of quantitatively analyzing a wafer of claim 1, wherein quantitatively analyzing the second surface of the wafer includes extracting the metal impurities from the second surface.

13. The method of quantitatively analyzing a wafer of claim 12, wherein extracting the metal impurities includes decomposing the second surface of the wafer and scanning an extraction solution droplet across the second surface.

14. The method of quantitatively analyzing a wafer of claim 12, wherein extracting the metal impurities includes adding an extraction solution droplet on the second surface and spreading the droplet across the second surface.

15. The method of quantitatively analyzing a wafer of claim 1, further comprising removing a native oxide layer from at least one of the first and second surfaces of the wafer.

16. The method of quantitatively analyzing a wafer of claim 15, wherein removing a native oxide layer includes removing the native oxide layer by vapor phase decomposition.

17. The method of quantitatively analyzing a wafer of claim 15, wherein removing a native oxide layer from at least one of the first surface and second surface of the wafer occurs before heating the first surface of the wafer.

18. A method for quantitatively analyzing a wafer for metal impurities, wherein the wafer has first and second surfaces, comprising:
removing a native oxide layer from at least one of the first and second surfaces of the wafer;
heating the first surface of the wafer via a hotplate to diffuse the metal impurities to the second surface in an environment at least substantially free of contamination, wherein a susceptor is used between the first surface of the wafer and the hotplate;
cooling the first surface of the wafer; and
quantitatively analyzing the second surface of the wafer for metal impurities.

19. The method of quantitatively analyzing a wafer of claim 18, wherein the metal impurities include nickel.

20. The method of quantitatively analyzing a wafer of claim 18, wherein removing a native oxide layer includes removing a native oxide layer by vapor phase decomposition.

21. The method of quantitatively analyzing a wafer of claim 18, wherein cooling the first surface of the wafer includes switching off the hotplate and leaving the first surface of the wafer in thermal communication with the hotplate.

22. The method of quantitatively analyzing a wafer of claim 18, wherein quantitatively analyzing the second surface of the wafer includes performing at least one of vapor phase decomposition inductively coupled plasma-mass spectrometry and vapor phase decomposition total x-ray fluorescence.

23. A method for quantitatively analyzing a wafer for nickel, wherein the wafer has first and second surfaces, comprising:
heating the first surface of the wafer to diffuse the nickel to the second surface in an environment at least substantially free of contamination;
cooling the first surface of the wafer; and
quantitatively analyzing the second surface of the wafer for the nickel.

24. The method for quantitatively analyzing a wafer of claim 23, wherein heating the first surface of the wafer includes heating the first surface of the wafer via a hotplate.

25. The method for quantitatively analyzing a wafer of claim 24, wherein heating the first surface of the wafer via a hotplate includes placing a susceptor between the first surface of the wafer and the hotplate.

26. The method of quantitatively analyzing a wafer of claim 24, wherein cooling the first surface of the wafer includes switching off the hotplate and leaving the first surface of the wafer in thermal communication with the hotplate.

27. The method of quantitatively analyzing a wafer of claim 23, wherein heating the first surface of the wafer includes heating the wafer in a non-oxygen containing environment.

28. The method of quantitatively analyzing a wafer of claim 23, wherein cooling the first surface of the wafer includes cooling the wafer via a cooling plate.

29. The method of quantitatively analyzing a wafer of claim 23, wherein cooling the first surface of the wafer includes cooling the wafer via a fluid solution.

30. The method of quantitatively analyzing a wafer of claim 23, wherein quantitatively analyzing the second surface of the wafer includes measuring the nickel on the second surface.

31. The method of quantitatively analyzing a wafer of claim 23, wherein quantitatively analyzing the second surface of the wafer includes extracting the nickel from the second surface.

32. The method of quantitatively analyzing a wafer of claim 23, further comprising removing a native oxide layer from at least one of the first and second surfaces of the wafer.

33. The method of quantitatively analyzing a wafer of claim 32, wherein removing a native oxide layer includes removing the native oxide layer by vapor phase decomposition.

34. The method of quantitatively analyzing a wafer of claim 32, wherein removing a native oxide layer from at least one of the first surface and second surface of the wafer occurs before heating the first surface of the wafer.
